(19) United States
(12) Patent Application Publication
(10) Pub. No.: US 2003/0100889 A1
(43) Pub. Date: May 29, 2003

(54) Method of administration of a gene of interest to a vascular tissue

(76) Inventors: Nicolas Duverger, Paris (FR); Didier Branellec, Lyon (FR); Nathalle Aubailly, Saint Medard en Jalles (FR); Glenda Bilder, Lederach, PA (US); Jeffrey Bostwick, Upper Black Eddy, PA (US)

Correspondence Address: Finnegan, Henderson , Farabow,, Garrett & Dunner, L.L.P., 1300 I Street, N.W., Washington, DC, 20005-3315, US

(21) Appl. No.: 10/187,797

(22) Filed: Jul. 3, 2002

Related U.S. Application Data
(60) Provisional application No. 60/302,658, filed on Jul. 5, 2001.

(51) Int. Cl. A61M 31/00

(52) U.S. Cl. 604/522; 604/509; 435/320.1

(57) ABSTRACT

The invention relates to a method for delivering a gene of interest to a vascular tissue comprising the following steps: (1) inserting into said vascular tissue a catheter in fluid communication with an inflatable balloon which is formed from a microporous membrane; and (2) delivering to said vascular tissue through the catheter a solution containing a vector comprising a gene of interest. This method can be used for the treatment of cardiovascular diseases, including hyperproliferative vascular disorders (such as restenosis), ischemic diseases (such as peripheral artery or coronary artery diseases), and atherosclerosis.

### DESCRIPTION OF THE INVENTION

This application claims the benefit of U.S. Provisional Application No. 60/302,658, filed Jul. 5, 2001, which is incorporated herein in its entirety.

The present invention relates to a method of administering a gene of interest to a vascular tissue (for example, an artery). In one embodiment of the invention, the vascular tissue may be an atherosclerotic artery, or a vascular tissue proximal to and connected with an ischemic tissue or an ischemic organ. The method comprises inserting into the vascular tissue an apparatus comprising a catheter and an inflatable balloon formed from a microporous membrane. In one embodiment, the invention relates to the treatment of cardiovascular diseases.

Percutaneous coronary interventions such as angioplasty, stent placement, or atherectomy are effective therapies for the treatment of myocardial infarction and ischemic heart disease. However, late reclosure or restenosis that occurs 6-12 months following these interventions remains a leading cause of revascularization failure, resulting in costly rehospitalization of 30-50% of patients for repeat procedures. The exact mechanism(s) of restenosis remains poorly understood, but smooth muscle cell (SMC) migration/proliferation/matrix production and vascular remodeling have been implicated in the process. More than 50 clinical trials have failed to identify an effective therapy for restenosis and, hence, novel therapies are clearly needed. Gene therapy offers a hope of success. Indeed, gene therapy targeted against the cellular processes of SMC migration and/or proliferation, or arterial remodeling, may provide an effective treatment for restenosis. However, to be effective, this therapy must consider not only the appropriate gene but also a means of optimal local delivery.

Local delivery of genes or gene products at the site of angioplasty, stent placement, or atherectomy may provide the means to precisely target the cells of interest and, thereby, achieve a higher concentration of the gene within the arterial wall than can be obtained by systemic administration. Furthermore, local catheter-based gene delivery would safeguard against systemic toxicity. The catheter must deliver the gene locally with precision and without leakage to the systemic circulation. Additionally, the catheter must be operator friendly.

Previous results have been obtained with various apparatuses, but none of them showed any convincing evidence of their ability to deliver a gene of therapeutic interest to atherosclerotic arteries. Specifically, the Infiltrator® catheter (Interventional Technologies) was used for adenoviral-mediated nitric oxide synthase (Varenne et al., Cir. 98:919-926, 1998, WO 98/34667) or C-type natriuretic peptide (Morishige et al., J. Amer. Coll. Cardio. 35:1040-1047, 2000) gene transfer to the injured coronary artery of the pig. However, the model system employed in these studies lacks relevance to humans and neither Varenne et al. nor Morishige et al. showed any evidence for the ability of the Infiltrator® catheter to deliver the adenoviral-gene complex to atherosclerotic arteries.

The Dispatch® catheter (Boston Scientific) was evaluated for adenoviral-mediated transfer of the marker gene beta-galactosidase (β-gal) in the normal and atherosclerotic iliac artery of the rabbit (Tahlil et al., Cardiovas. Res. 33:1810-187, 1997). Although local delivery of the marker gene was achieved, there was evidence of viral leakage, which raised the possibility of systemic toxicity with use of the Dispatch® catheter. The performance of the Dispatch® catheter in a coronary artery was not evaluated. Additionally, the hydrogel-coated balloon catheter (Slider with Hydroplus®, Mansfield Medical, Boston Scientific Corporation) was used to transfer adenoviral β-gal constructs to the normal rabbit iliac artery (Steg et al., Cir. 90:1648-1656, 1994), and the channeled balloon catheter (Palasis et al., Hum. Gene Ther. 11:237-246, 2000; Boston Scientific Corporation) was successfully used to transfer the adenoviral Gax gene to the normal rabbit iliac artery simultaneously with balloon angioplasty (Maillard et al., Cardiovas. Res. 35:536-546, 1997).

A recent report of local delivery devices compared the Infiltrator, Crescendo®, Infusasleeve®, and channeled balloon catheters in the porcine coronary artery (Varenne et al. Hum. Gene. Ther. 10:1105-1115, 1999). In this study, the authors showed that higher transduction levels were achieved in arteries with the Infiltrator®, Crescendo®, and Infusasleeve® catheters compared with the channeled balloon catheter. Nevertheless, these apparatuses have not shown any convincing evidence of their ability to deliver a gene of therapeutic interest to atherosclerotic arteries. Microporous balloons have been tested, according to Application WO 98/34667, but the use of these balloons would result in “limited transgene expression”. Other apparatuses comprising catheters have been described with regard to their use in delivery of drugs. However, the possibility of using them to transfer genes of interest to the arterial wall has never been disclosed.

### SUMMARY OF THE INVENTION

One object of the present invention is to provide a method for delivering a gene of interest to a vascular tissue comprising the following steps: (1) inserting into said vascular tissue a catheter in fluid communication with an inflatable balloon formed from a microporous membrane, and (2) delivering to said vascular tissue through the catheter a solution containing a vector comprising a gene of interest, wherein said gene of interest is expressed in said vascular tissue. In one embodiment, the vector is a viral vector, for example, an adenoviral vector. In one embodiment of the invention, the balloon is inflated, the microporous membrane has pores with an approximate size from about 10 Å to about 1μ and a pore density from about 10⁴ pores/cm² to about 10¹¹ pores/cm², and the vascular tissue is an artery. In one aspect of said method, the vascular tissue is an atherosclerotic artery, or a vascular tissue proximal to and connected with an ischemic tissue or an ischemic organ.

Another aspect of the present invention is a method for treating cardiovascular disease comprising the following steps: (1) inserting into a vascular tissue a catheter in fluid communication with an inflatable balloon formed from a microporous membrane, and (2) delivering to said vascular tissue through the catheter a solution containing a vector comprising a gene of interest, wherein said gene of interest is expressed in said vascular tissue. Said method can be used for treating restenosis and comprises the following steps: (1) inserting into an atherosclerotic artery a catheter in fluid communication with an inflatable balloon formed from a microporous membrane, and (2) delivering to said artery through the catheter a solution containing a vector comprising a suicide gene, wherein said gene is expressed in said atherosclerotic artery. Said suicide gene may, for example, be a thymidine kinase gene or a cytosine deaminase gene. Said method can also be used for treating an ischemic tissue or an ischemic organ and comprises the following steps: (1) inserting into a vascular tissue proximal to and connected with said ischemic tissue or ischemic organ a catheter in fluid communication with an inflatable balloon formed from a microporous membrane, and (2) delivering to said vascular tissue through the catheter a solution containing a vector comprising a gene encoding an angiogenic factor, wherein said gene is expressed in said vascular tissue. Said angiogenic factor may, for example, be fibroblast growth factor (FGF) or vascular endothelial growth factor (VEGF).

Another object of the invention is the use of a vector comprising a gene of interest, wherein said gene of interest is expressed in a vascular tissue, for the delivery of a gene of interest to said vascular tissue by intravascular administration through an inflatable balloon formed from a microporous membrane.

A further object of the invention is the use of a vector comprising a gene of interest, wherein said gene of interest is expressed in a vascular tissue, for the manufacture of a pharmaceutical composition for use in the treatment of vascular diseases by intravascular administration through an inflatable balloon formed from a microporous membrane. Said disease can, for example, be restenosis or an ischemic disease. Said gene can, for example, be a suicide gene, such as a thymidine kinase gene or a cytosine deaminase gene, or a gene encoding an angiogenic factor such as FGF or VEGF.

Another object of the invention is an apparatus or a kit comprising: (1) a catheter in fluid communication with an inflatable balloon formed from a microporous membrane, and (2) a vector comprising a gene of interest. Said apparatus can be used in the treatment of, for example, cardiovascular disease.

### DESCRIPTION OF THE DRAWINGS

FIG. 1: Adenovirus-mediated gene transfer to atheromatous rabbit iliac artery using a channeled balloon delivery device (X30).

FIG. 2: Adenovirus-mediated gene transfer to atheromatous rabbit iliac artery using a channeled balloon delivery device (X40).

FIG. 3: Adenovirus-mediated gene transfer to atheromatous rabbit iliac artery using a microporous delivery device (e-Med Corporation) (X30).

FIG. 4: Adenovirus-mediated gene transfer to atheromatous rabbit iliac artery using a microporous delivery device (e-Med Corporation) (X40).

FIG. 5: Intensity of apparent β-gal staining. N=6 (left anterior descending coronary artery (LAD)) & 6 (left circumflex coronary artery (LCX)) for the annular and IVT Infiltrator® groups; N=8 (LAD) & 8 (LCX) for the microporous (e-Med Corporation) and channeled groups. Data are the mean±the standard error of the mean (sem).

FIG. 6: Area of apparent β-gal staining in the LAD & LCX coronary arteries. N=6 (LAD) & 6 (LCX) for the annular and IVT Infiltrator® groups; N=8 (LAD) & 8 (LCX) for the microporous (e-Med Corporation) and channeled groups. Data are the mean±sem.

FIG. 7A: Average total serum cholesterol at the time of balloon over-stretch and gene transfer. N=30. *p<0.05 (Student's t-test).

FIG. 7B: Average total serum cholesterol level analyzed by treatment group. N=6 for the annular and IVT Infiltrator® groups; N=8 for the microporous (e-Med Corporation) and channeled groups. *p<0.05 vs. e-Med Corporation and channeled; **p<0.05 vs. channeled analysis of variance (ANOVA). Data are the mean±sem.

FIG. 8: Delivery device balloon diameter to lumen diameter ratios. N=6 (LAD) & 6 (LCX) for the annular and IVT Infiltrator® groups; N=8 (LAD) & 8 (LCX) for the microporous (e-Med Corporation) and channeled groups. Data are the mean±sem.

FIG. 9: Number of branches per treated segment of artery. N=27 (LAD) & 26 (LCX). p<0.05 (Student's t-test). Data are the mean±sem.

FIG. 10: Adenovirus-mediated gene transfer to porcine coronary artery using a channeled balloon delivery device.

FIG. 11: Adenovirus-mediated gene transfer to porcine coronary artery using a microporous (e-Med Corporation) balloon delivery device.

### DETAILED DESCRIPTION

The Microporous Catheter

According to the present invention, the vector comprising a gene of interest is administered to an arterial cell by intraarterial administration through an inflatable balloon formed from a microporous membrane. In one embodiment, this microporous catheter is the one described in U.S. Pat. No. 5,569,198 and PCT Application WO 96/22805, which are incorporated by reference. The use of microporous catheters to deliver compounds that reduce vasomotor activity (e.g., calcium channel antagonists) and inflammatory responses is described, but not the use of these catheters to deliver vectors (especially, viral vectors). According to one embodiment, said apparatus is the Microfuse® infusion catheter (e-Med Corporation, St. Paul, Minn.). This catheter is intended for localized delivery of solutions in the coronary arteries. Such an apparatus is typically composed of two parts: the catheter part (strictly speaking) and the inflatable part formed from a porous membrane (i.e., the balloon). Both parts are in fluid communication through the lumen of the catheter.

The design of the microporous catheter according to the present invention permits appropriate contact of the balloon with the arterial wall such that vector-mediated gene transfer can occur within 45 seconds. In one embodiment, the pore size of the membrane is between about 0.05μ and about 1μ and the pore density is between about 10⁶ pores/cm² and about 10⁹ pores/cm². In another embodiment, the pore size of the membrane is about 1μ and the pore density is about 10⁶ pores/cm². However, the microporous catheter according to the present invention may be modified by an increase in the number of pores, a change in pore diameter, addition of a flow-through partition, or strengthening the balloon to enable stent placement. These changes may serve to accommodate other future vectors, enhance the quantity of gene transferred, and/or reduce procedure time by combining gene transfer with stent placement.

The microporous catheter is used as described in U.S. Pat. No. 5,569,198 and PCT Application WO 96/22805, or according to the manufacturer's instructions. The guide wire is first inserted into the selected artery to a point past the desired position in the arterial system where the administration of vector should occur. The catheter including the catheter body and the microporous balloon is then advanced along the guide wire to this desired position so that the microporous balloon is aligned with the target region of the artery. The microporous balloon is then inflated by introducing an inflation fluid through the balloon lumen into the chamber. During inflation, the outer surfaces of the microporous balloon press outwardly against the inner surfaces of the vessel wall. Application of a predetermined pressure results in delivery of the solution containing the vector and transformation of the cells surrounding the vessel. The pressure inside the balloon is not great enough to cause more than a minimal amount of agent to escape from the balloon until the microporous balloon is in contact with the wall of the vessel. Such a pressure is between about 0.5 and 20 atmospheres (atm) (i.e., between about 7 and 294 pounds per square inch). It should be such as not to induce any alteration of the arterial wall. In one embodiment, such a pressure is about 1 to 5 atm. After delivery of the agent is complete, the microporous balloon is deflated and either removed from the patient's body or placed at a different location for treatment of another position.

Vascular Tissue

A vascular tissue means any tissue within an internal tubular structure called a vessel that is connected to a tissue or organ within the body of a mammal (including a human). Within the cavity of the tubular structure, a bodily fluid flows to or from the body part. Examples of bodily fluid include blood or lymphatic fluid. Examples of vessels include arteries, arterioles, venules, sinusoids, veins, and lymphatics. The intravascular route includes delivery through the blood vessels such as an artery or a vein.

Afferent blood vessels of organs are defined as vessels in which blood flows toward the organ or tissue under normal physiological conditions. Efferent blood vessels are defined as vessels in which blood flows away from the organ or tissue under normal physiological conditions. In the heart, afferent vessels are known as coronary arteries, while efferent vessels are referred to as coronary veins. In one embodiment, the vascular tissue is an artery, and in particular a coronary artery.

The Vectors

The vectors according to the present invention can be viral vectors or nonviral vectors, such as liposome-based plasmid delivery systems or synthetic virus-like systems. In one embodiment, the vectors according to the present invention are viral vectors. Among the later, there may be mentioned, inter alia, recombinant adenoviruses, recombinant adeno-associated viruses, recombinant retroviruses (such as Moloney Leukemia Virus-derived viruses), lentiviruses, herpesvirus, vaccinia virus, and hybrid viral vectors whose preparation may be carried out according to methods known to persons skilled in the art. Chimeric viral vectors may, for example, be the adenovirus-retrovirus chimeric vectors, which are described inter alia in PCT Application WO 95/22617, or the episome/adenovirus vectors which are described by Leblois et al., Mol. Ther. 1:314-322, 2000 and in PCT Application WO 97/47757. In one embodiment, the viral vectors are adenoviral vectors.

The Adenoviral Vectors

In one embodiment, adenoviral vectors used according to the present invention are vectors derived from defective adenoviruses, that is to say that they are incapable of autonomously replicating in the target cell. The construction of these defective viruses as well as their infectious properties has been widely described in the literature (see in particular S. Baeck and K. L. March, Circul. Res., 82:295-305, 1998; Shenk et al., Adenoviridae: Viruses and Replication (in Virology), 211-2148, EDS—Ravens Publishers, Philadelphia, 1996; Yeh et al., FASEB J., 11:615-623, 1997).

Various adenovirus serotypes, whose structure and properties vary somewhat, have been characterized. Among these serotypes, use may be made in the context of the present invention of the type 2 or 5 human adenoviruses (Ad2 or Ad5), adenoviruses of animal origin such as those described in Application FR 93 05954, or adenoviruses of mixed origin. Among the adenoviruses of animal origin which can be used in the context of the present invention, there may be mentioned the adenoviruses of canine, bovine, murine (Beard et al., Virology, 75:81, 1990), ovine, porcine, avian, or simian origin. In one embodiment, the adenovirus of animal origin is a canine adenovirus, for example, a CAV2 adenovirus (Manhattan or A26/61 strain) as described in Application WO 94/26914. The defective adenoviruses of the invention comprise, in general, an inverted terminal repeat (ITR) at each end, a sequence allowing encapsidation (Psi), the E1 gene, and at least one of the genes E2, E4 and L1-L-5 having been inactivated by any technique known to persons skilled in the art (Levero et al., Gene, 101:195, 1991; EP 185,573; Graham, EMBO J., 3:2917, 1984). In one embodiment, the recombinant adenovirus used in the context of the invention comprises a deletion in the E1 region of its genome. In another embodiment, the recombinant retrovirus comprises a deletion of the E1a and E1b regions. There may be mentioned, for example, deletions affecting nucleotides 454-3328, 382-3446 or 357-4020 (with reference to the genome of Ad5).

According to one variant, the recombinant adenovirus used in the context of the invention comprises, in addition, a deletion in the E4 region of its genome. In another embodiment, the deletion in the E4 region affects all the open reading frames. There may be mentioned, by way of example, the 33466-35535 or 33093-35535 deletions. Other types of deletions in the E4 region are described in Applications WO 95/02697 and WO 96/22378, which are incorporated herein by reference.

In another embodiment, the adenovirus fiber protein or the hexon protein has been modified to permit effective targeting of these adenoviral vectors. For example, the HRV5 loop of the hexon protein or the Hi loop (knob) of the fiber protein can be modified as disclosed in Application WO 00/12738. Such vectors can be modified for targeting urokinase-type plasminogen activator receptor-bearing cells.

In general, the recombinant adenoviruses according to the invention are formulated and administered in the form of doses of between 10⁴ and 10¹⁴ pfu. In one embodiment, the recombinant adenoviruses according to the invention are formulated and administered in the form of doses of between 10⁸ and 10¹² pfu. The term pfu (plaque forming unit) corresponds to the infectious power of a viral solution, and is determined by infecting an appropriate cell culture, and measuring the number of plaques of infected cells. The techniques for determining the pfu titer of a viral solution are well known in the art. The viral solution may contain any pharmaceutically acceptable carrier, vehicle, or excipient, such as saline buffer, isotonic solution, additives, stabilizers, etc.

Other Viral Vectors

As regards the adeno-associated viruses (AAV), they are relatively small-sized DNA viruses that integrate into the genome of the cells that they infect, in a stable and site-specific manner. They are capable of infecting a broad spectrum of cells, without inducing any effect on cell growth, morphology or differentiation. Moreover, they do not appear to be involved in pathologies in humans. The AAV genome has been cloned, sequenced, and characterized. It comprises about 4,700 bases and contains, at each end, an inverted terminal repeat (ITR) of about 145 bases, serving as a replication origin for the virus. The remainder of the genome is divided into two essential regions carrying the encapsidation functions: the left portion of the genome, which contains the rep gene involved in viral replication and the expression of the viral genes, and the left portion of the genome, which contains the cap gene encoding the virus capsid proteins.

The use of AAV-derived vectors for the transfer of genes in vitro and in vivo has been described (see in particular WO 91/18088, WO 93/09239, U.S. Pat. No. 4,797,368, U.S. Pat. No. 5,139,941, and EP 488528). These applications and patents describe various AAV-derived constructs in which the rep and/or cap genes have been deleted and replaced with a gene of interest, and their use for transferring in vitro (to cells in culture) or in vivo (directly in an organism) the gene of interest. The replication-defective recombinant AAVs according to the invention may be prepared by co-transfection, into a cell line infected with a human helper virus (for example, an adenovirus), of a plasmid containing the nucleic sequences of the invention bordered by two AAV inverted terminal repeats (ITR), and of a plasmid carrying the AAV encapsidation genes (rep and cap genes). The recombinant AAVs produced are then purified by conventional techniques.

Lentiviruses may also be used according to this embodiment; and they allow the transfer and efficient and stable integration of a gene of interest into quiescent cells. There may be mentioned, for example, human T-lymphotropic virus-1 (HTLV-1), or animal lentiviruses such as feline immunodeficiency virus (FIV), equine infectious anemia virus (EIAV; WO 98/51810), bovine immunodeficiency virus (BIV), simian immunodeficiency virus (SIV), caprine arthritis encephalitis virus (CAEV; WO 98/39463; Naldini et al., Science 272:263-267, 1996; Schnele et al., Hum. Gen. Ther. 11:439-447, 2000), or a lentivirus related to the one which causes AIDS, i.e., human immunodeficiency virus-2 (HIV-2) which is not highly pathogenic in humans (Kundra et al., Hum. Gen. Ther. 9:1371-1380, 1998.

The Gene of Interest

The microporous catheter according to the present invention could be used for delivery of a variety of genes including those that inhibit proliferation, migration and/or matrix production of smooth muscle cells, those that inhibit focal shrinkage, those that inhibit macrophage infiltration and production of cytokines, those that retard the thrombogenicity of the arterial wall, those that improve vasorelaxation, those that improve collateral arterial production, and those that prevent lipid uptake and/or oxidation.

In one embodiment, the gene of interest encodes a protein or an RNA that may be involved in cardiac pathologies such as cardiac insufficiency, cardiac hypertrophy, hypoxia, ischemia, or cardiac transplant rejection.

As proteins of therapeutic interest, there may be mentioned, inter alia:

proteins inducing angiogenesis, such as members of the VEGF family (including VEGF-A, VEGF-B, VEGF-C, and VEGF-D), members of the FGF family (including FGF1, FGF2, FGF4, and FGF5), angiogenin, endothelial growth factor (EGF), transforming growth factors alpha and beta (TGFα and TGFβ), tumor necrosis factor alpha (TNFα), developmental endothelial locus-1 (Del-1), Scatter Factor/hepatocyte growth factor (HGF), members of the angiopoietin family, cytokines including interleukins (IL) (including IL-1, IL-2, and IL-8), angiotensin-2, tissue plasminogen activator (TPA), urokinase (uPA), human tissue kallikrein, the molecules involved in the synthesis of active lipids (prostaglandins, cyclooxygenase-1), factors inducing the production of angiogenic growth factors such as hypoxia-inducible factor (HIF), and protein kinases such as protein kinase B/Akt (AKT/PKB);

proteins involved in the control of cardiac contractility, such as phospholamban, phospholamban inhibitors, sarcoplasmic/endoplasmic reticulum Ca²⁺ pump (SERCA-2a), β2-adrenergic receptor, or dystrophin or minidystrophin (FR 91 11947);

proteins with cytoprotective activity, which, for example, block apoptosis, such as proteins which are members of the B-cell leukemia/lymphoma (bcl) family, and protein kinases such as AKT/PKB;

transcription factors, such as natural or chimeric nuclear receptors, comprising a DNA-binding domain, a ligand-binding domain and a transcription activating or inhibiting domain, such as, for example, the fusion proteins tetR-NLS-VP16, the fusion proteins derived from estrogen receptors, the fusion proteins derived from steroid hormone receptors, the fusion proteins derived from progesterone receptors, the proteins of the Chemical Inducer of Dimerization (CID) system described by Rivera et al., Nature Medicine, 2:1028-1032, 1996. There may be mentioned, for example, as chimeric nuclear receptors, the nuclear receptors Peroxisome Proliferator Activated Receptor (PPAR) and PPARγ2, as described in Applications WO 96/23884 and FR 99 07957, and by Frohnert et al., J. Biol. Chem., 274:3970-3977, 1999, and Mukherjee et al., J. Biol. Chem., 272:8071-8076, 1997 either in its native form, without modification of the primary structure, or a modified PPARγ2 comprising one or more ligand-binding sites or E/F domains (Schoonjans et al., Biochim. Biophys. Acta., 1302:93-109, 1996, such as PPARγ2γ2;

immunosuppressors such as, for example, interleukins 2 and 10, which make it possible to completely or partially inhibit an immune signaling pathway and thus to extend the duration of cardiac transplants;

proteins involved as agents for reducing hypoxia such as nitric oxide synthase (NOS), B-cell leukemia/lymphoma 2 (bcl-2), superoxide dismutase (SOD), and catalase.

As RNAs of therapeutic interest, there may be mentioned, for example, antisense RNAs which are useful for controlling the expression of genes or the transcription of cellular mRNAs, thus blocking translation into a protein according to the technique described in EP 140,308, as well as the ribozymes which are capable of selectively destroying target RNAs as described in EP 321,201.

It is understood that the present invention is not limited to the specific examples of proteins or RNAs, but that it can be used by persons skilled in the art for the expression of any nucleic acid in, for example, myocardial and vascular cells, by simple customary experimentation.

The gene of interest may be placed under the control of any suitable transcriptional promoter. In one embodiment, the transcriptional promoter is functional in mammalian cells (for example, human cells). The promoter may be constitutive, ubiquitous, regulated, inducible, tissue-specific, etc. The choice of a particular promoter can be made by the skilled person, depending on the disease to be treated.

Specific examples of promoters suitable for use in the present invention include promoters of viral or mammalian origin (such as cytomegalovirus, respiratory syncitial virus, Rous sarcoma virus, simian virus 40, retrovirus, lentivirus, and the like known in the art), housekeeping promoters (such as EF1alpha or E2F) or specifically-expressed promoters (such as VEGF(s), FGF(s), HIF(s), Tie, vascular endothelium cadherin, creatine kinase M, desmin, phosphoglycerate kinase (PGK), alpha smooth muscle cell actin, SM22, steel, kit, cardiac myosin, alpha myosin heavy chain, neuronal-specific promoters, and all promoters from all similar genes known by persons skilled in the art). Promoters could also be of synthetic origin such as inducible or regulated promoters (modulated by tetracycline, rapamycin, PPAR activator, hormone analogs, pathophysiological situations, etc.) or any promoters derived from fragments of the above using techniques known by the skilled person (deletion mutants, chimeric promoter, etc.).

Diseases to be Treated

The method according to the present invention can be used for the treatment of any disease that is susceptible to being cured or prevented by administering to a vascular tissue a vector (for example, an adenoviral vector) encoding a gene of therapeutic interest. The tissue to be treated can be a vascular tissue but it can also be a nonvascular tissue proximal to and connected with this vascular tissue. Such a nonvascular tissue can be a muscle tissue (for example, a myocardial tissue or a skeletal muscle tissue).

Thus, the present method is intended for the treatment of cardiovascular diseases. In one embodiment, the present method is intended for the treatment of hyperproliferative vascular disorders, such as restenosis. It is also adapted for the treatment of ischemic diseases, such as peripheral artery disease or coronary artery disease. In one embodiment, the method according to the present invention comprises stimulating collateral vessel development in patients having myocardial ischemia. Thus, the method allows the treatment of diseases directly linked to vascular tissues and diseases linked to ischemic tissues.

For the treatment of diseases linked to an organ or tissue other than vascular tissues, the vector is administered into a vascular tissue in proximity to the organ or tissues to be treated. Thus, the present method allows the treatment of any organ near vascular tissues or of any vascularized organ.

In one embodiment, the methods according to the present invention comprise a step of infusing an adenoviral vector into a blood vessel that perfuses the heart. The present invention may be used for the treatment of humans. However, it can be adapted for the treatment of any animal (for example, any mammal).

Treatment of Restenosis

In one embodiment, the method according to the present invention is applied to the treatment of restenosis. In such a case, an adenovirus comprising a suicide gene can be used. According to the present invention, a suicide gene is a gene whose expression renders the infected cell sensitive to a therapeutic agent. For example, one suicide gene is the thymidine kinase gene, which renders cells sensitive to agents such as ganciclovir and acyclovir. Another suicide gene is the cytosine deaminase gene, which renders cells sensitive to 5-fluorocytosine (5-FC).

In one embodiment, the thymidine kinase gene of the human herpes virus (hTK HSV-1) is used. The sequence of this gene has already been disclosed (in particular by McKnight et al., Nucleic Acids Res. 8:5931, 1980). It is also possible to use derivatives of this sequence having greater substrate specificity or better kinase activity. Such derivatives may be obtained, for example, by mutagenesis as described by Balasubramaniam et al., J. Gen. Virol., 71:2979, 1990 and Munir et al., J. Biol. Chem., 267:6584, 1992.

It is also possible to use the cytosine deaminase gene, which confers sensitivity to 5-fluorocytosine (5-FC). The cells that express this gene are able to convert 5-fluorocytosine (5-FC) to 5-fluorouracil (5-FU), which is a toxic metabolite. The sequence of this gene has been disclosed by Anderson et al., Arch. Microbiol., 152:115, 1989.

An adenovirus comprising a suicide gene can, for example, be the adenovirus Ad-RSV-tk, the construction of which is described in Application WO 96/05321 (Rhône Poulenc Rorer).

To carry out the present invention, a person skilled in the art can refer to Sambrook et al. (Molecular Cloning, A Laboratory Manual, Cold Spring Harbor Laboratory Press, New York 1989).

The following examples illustrate particular embodiments of the present invention and are not intended to limit the specification and claims in any way.

### EXAMPLE 1

### Adenoviral-Mediated β-Gal Gene Transfer in the Rabbit Iliac Artery Model of Stenosis

1. Summary

The ability of local delivery devices to deliver a marker gene, β-gal, encoded by an adenovirus (AV_{1.0} cytomegalovirus (CMV) β-gal/10¹² viral particles per artery) to the rabbit iliac artery of normal and hypercholesterolemic animals was evaluated. Devices used for local delivery were an e-Med catheter, a channeled balloon catheter, and an annular balloon catheter. Gene transfer efficacy was determined by microscopic analysis of cells staining for β-gal. Data indicated greater efficiency, higher cellular transduction, and homogeneity over the artery segment with the e-Med catheter in comparison with that obtained with the two other devices.

2. Material and Methods

2.1. Material and Methods for Gene Transfer in Normal Rabbits

2.1.1 Animals

Species: Rabbits.

Strain: New Zealand White.

Sex: Male.

Characterization: Normal.

Body weight range at initiation of treatment: 4.0 kg.

2.1.2. Treatment

Adenovirus description

| Construct | Batch | Particle number |
|---|---|---|
| AV_{1.0} CMVβ-gal | V143 | 5 × 10¹² vp/ml |

Buffer: PBS containing 10% glycerol

Storage: −80° C.

Adenovirus Preparation

PBS solution was prepared as follows:

| | | |
|---|---|---|
| DPBS pH 7.4 | 45 | ml |
| Glycerol 10% | 5 | ml |
| MgC1₂ 0.5 M 0.1% | 50 | μl |

Adenoviral aliquots were rapidly thawed at room temperature and diluted in the PBS solution described above. Dilutions were prepared just before use to avoid any virus degradation with prolonged storage.

Animal Groups and Treatment

At Day 0:

Rabbits were weighed.

Anesthesia Step:

Following placement in the restraining cage and after disinfection, an intramuscular injection of a mixture of ketamine (50 mg/kg)/Xylazine (5 mg/kg) was administered. A 22-gauge catheter was placed in the marginal ear vein and was flushed with sodium heparinate (120 IU/kg). Blood samples were taken.

Injury step for annular, channeled and e-Med Microfuse® balloon catheter gene transfer delivery:

A 5-F introducer sheath (Vygon) was positioned in the carotid artery. All catheters were subsequently introduced through this sheath.

A 3 cm-long, 2.5 mm angioplasty balloon (Nycomed) was then advanced over a 0.014″ guide wire (Boston Scientific). Balloon inflation was performed 3 times for 1 minute each at 6 atm (nominal pressure). The balloon was deflated for 1 minute between inflations. After this initial injury, the same balloon was used to perform the injury on the contralateral external iliac artery in a similar fashion.

Infection Step:

1. A 3-way stopcock was attached to each end of the flexible extension tubing.

2. The Leveen Inflator® was filled with a 50/50 mixture of saline and contrast agent. The inflation device was purged of air, and was attached to the flexible extension tubing via one of the stopcocks.

3. The stopcock was attached at the free end of the flexible extension tubing to the INF port on the catheter.

4. The proximal stopcock was set to allow filling of the flexible extension tubing with the desired volume of drug solution.

5. With the distal stopcock open to air, and the proximal stopcock set to allow connection between the infusion device and the extension tubing, the filling operation within the flexible extension tubing was completed using solution from the infusion device.

6. The distal stopcock was closed to air, thereby allowing connection between the extension tubing and the catheter.

7. Prior to inserting the guide wire into the e-Med Microfuse®, annular, or channeled balloon catheters, the wire lumen of the catheter was flushed with sterile saline using a 20 ml syringe.

8. The balloon was introduced via the carotid into the external iliac artery immediately distal to the origin of the internal iliac artery under radioscopic guidance.

9. Air (equivalent to the dead space, approximately 900 μl) and the viral solution (500 μl) were chased using a Leveen Inflator® with a 50/50 mixture of saline and contrast agent.

10. The pressure was monitored and kept at 3 atm.

11. The balloon was deflated when contrast dye in the balloon appeared. Therefore, infusion time fluctuated from one rabbit to another.

12. The balloon was introduced in the contralateral iliac artery.

13. A new extension tube was filled with viral solution and the procedure was repeated.

14. After the second transfer, the balloon was deflated and removed.

15. The carotid artery was ligated with 3.0 silk (Ethicon).

16. The wound was closed with one subcutaneous suture (4.0 silk) and a cutaneous suture (2.0 suturamid). The animals were allowed to recover from anesthesia and returned to their cage.

All animal deaths occurring during the experiment were recorded.

2.1.3 Examinations Performed

Three days after transfer, animals were killed by pentobarbital overdose. The treated arterial segments were harvested for histochemical analyses and dipped in 3.7% Formalin solution. At this step, samples were transferred out of the L2 laboratory to the anatomic histology unit.

2.2. Material and Methods for Gene Transfer in Atheromatous Rabbits

2.2.1 Animals

Species: Rabbits.

Strain: New Zealand White.

Sex: Male.

Characterization: Normal

Body weight range at initiation of treatment: 4.0 kg.

2.2.2 Treatment

Adenovirus description

| Construct | Batch | Particle number | Titer |
|---|---|---|---|
| AV_{1.0} CMVβ-gal | V120 | 4.1 × 10¹² vp/ml | 4.5 × 10¹⁰ pfu/ml |

Buffer: PBS containing 10% glycerol

Storage: −80° C.

Adenovirus Preparation

PBS solution was prepared as follows:

| | | |
|---|---|---|
| DPBS pH 7.4 | 45 | ml |
| Glycerol 10% | 5 | ml |
| MgCl₂ 0.5 M 0.1% | 50 | μl |

The adenoviral aliquots were rapidly thawed at room temperature and diluted in the PBS solution described above. Dilutions were prepared just before use to avoid any virus degradation with prolonged storage.

Animal Groups and Treatment

Adenovirus was administered as described in the following table:

| | | Injected dose | Infusion | Pure adeno | PBS volume |
|---|---|---|---|---|---|
| Group | n | (vp/artery) | time | volume (μl) | (μl) |
| 1 | 6 | 1 × 10¹² | 2 min | 244 | 256 |
| 2 | 4 | 0 | 2 min | 0 | 500 |

At Day 1:

Rabbits were weighed and blood samples were taken.

Animals were fed 140 g daily with a 1.33% cholesterol diet provided by U.A.R. (Usine d'Alimentation Rationelle. 7,rue Galliéni. Villemoison. 91360 Epinay sur Orge).

At Day 8:

Blood samples were taken.

At Day 15: Injury Step:

Blood samples were taken.

Following placement in the restraining cage and after disinfection, an intramuscular injection of a mixture of Ketamine (50 mg/kg)/Xylazine (5 mg/kg) was administered. A 22-gauge catheter was placed in the marginal ear vein and was flushed with sodium heparinate (120 IU/kg). Blood samples were taken.

A 5-F introducer sheath (Vygon) was positioned in the carotid artery. All catheters were subsequently introduced through this sheath.

A 3 cm-long, 2.5 mm angioplasty balloon (Nycomed) was then advanced over a 0.014″ guide wire (Boston Scientific). Balloon inflation was performed 3 times for 1 minute each at 6 atm (nominal pressure). The balloon was deflated for 1 minute between inflations. After this initial injury, the same balloon was used to perform the injury on the contralateral external iliac artery in a similar fashion.

At Day 29:

Blood samples were taken.

At Day 43 (4 Weeks After the Initial Abrasion): Infection Step:

Blood samples were taken.

Following placement in the restraining cage and after disinfection, an intramuscular injection of a mixture of Ketamine (50 mg/kg)/Xylazine (5 mg/kg) was administered. A 22-gauge catheter was placed in the marginal ear vein and was flushed with sodium heparinate (120 IU/kg). Blood samples were taken.

A 5-F introducer sheath (Vygon) was positioned in the carotid artery. All catheters were subsequently introduced through this sheath.

A 2 cm-long, 2.5 mm channeled balloon catheter (Boston Scientific) or e-Med catheter was used for the transfer. 350 μl of the viral solution were infused in the catheter (equivalent to the predetermined dead-space).

The catheter was then advanced over a 0.014″ guide wire and inflated with contrast medium at 6 atm 3 times for 1 minute each to perform the second injury. The catheter was then inflated at the same pressure and the viral solution was chased using a syringe pump with 550 μl of contrast solution.

The infusion time was 2 min. The pressure was monitored and recorded with a pressure transducer.

The balloon was deflated, placed in the contralateral artery, and the procedure was repeated.

The wound was closed with one subcutaneous suture (4.0 silk) and a cutaneous suture (2.0 suturamid). The animals were allowed to recover from anesthesia and returned to their cage.

At Day 46:

All animals were sacrificed.

All animal deaths occurring during the experiment were recorded.

2.2.3 Examinations Performed

Post Mortem Procedure

Three days after transfer, animals were killed by pentobarbital overdose. The treated arterial segments were harvested for histochemical analyses and dipped in 3.7% Formalin solution. At this step, samples were transferred out of the L2 laboratory to the anatomic histology unit.

2.3 Histological Analysis

After X-gal incubation, arterial segments were maintained in a 3.7% formalin solution until paraffin embedding. Sections (5 μm) for histochemical analysis were routinely processed with Nuclear Red as counterstaining. Macroscopic β-gal expression (“blue arteries”) and microscopic β-gal expression were also studied in the arterial wall.

3. Results

Summary of the Results in Normal Rabbits

Thirty-three percent of iliac arteries (6/18 arteries) were found having more than 120 β-gal positive cells in 4 sections using the e-Med catheter versus only 22 (2/9 arteries) and 12% (1/8 arteries) of arteries with the annular balloon and channeled balloon catheters, respectively. Thirty-three percent of iliac arteries (6/18 arteries) exhibited between 30 and 120 β-gal positive cells in 4 sections using the e-Med catheter versus only 22 (2/9 arteries) and 12% (1/8 arteries) of arteries with the annular balloon and channeled balloon catheters, respectively. Only 33% of iliac arteries (6/18 arteries) displayed less than 30 β-gal positive cells in 4 sections using the e-Med catheter, whereas there were 55% (5/9 arteries) and 75% (6/8 arteries) of arteries with the annular balloon and channeled balloon catheters, respectively. These data indicated superior gene transfer into the arterial wall using the e-Med microporous catheter.

Summary of the Results in Atheromatous Rabbits

In this model of athersclerosis, more than 71% of iliac arteries (10/14 arteries) displayed more than 120 β-gal positive cells in 4 sections using the e-Med catheter, whereas no staining was observed (0/8 arteries) in arteries transduced with the use of the channeled balloon catheters. Fourteen percent of iliac arteries (2/14 arteries) showed between 30 and 120 β-gal positive cells in 4 sections using the e-Med catheter (50% for the channeled balloon catheter). Only 14% of iliac arteries (2/14 arteries) exhibited less than 30 β-gal positive cells in 4 sections using the e-Med catheter, applied to 50% (3/6 arteries) arteries the use the channeled balloon catheter. Again, these data indicated superior gene transfer in the arterial wall using the e-Med microporous catheter. In addition, gene transfer was homogenous along the transfer length (balloon length was 20 mm). The mean lengths of gene transfer were 14.6 ±1.1 mm (n=14) and <4 mm (n=8) using e-Med and channeled balloon catheters, respectively (p<0.05; ANOVA).

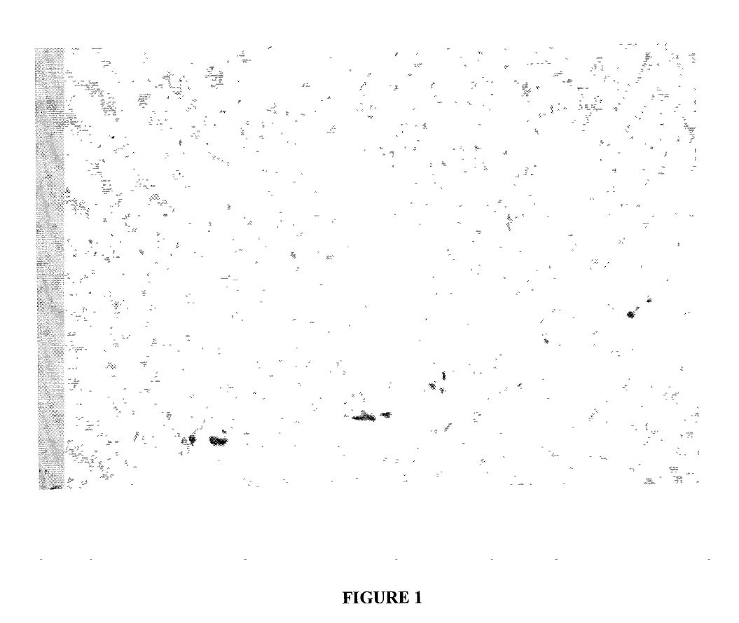

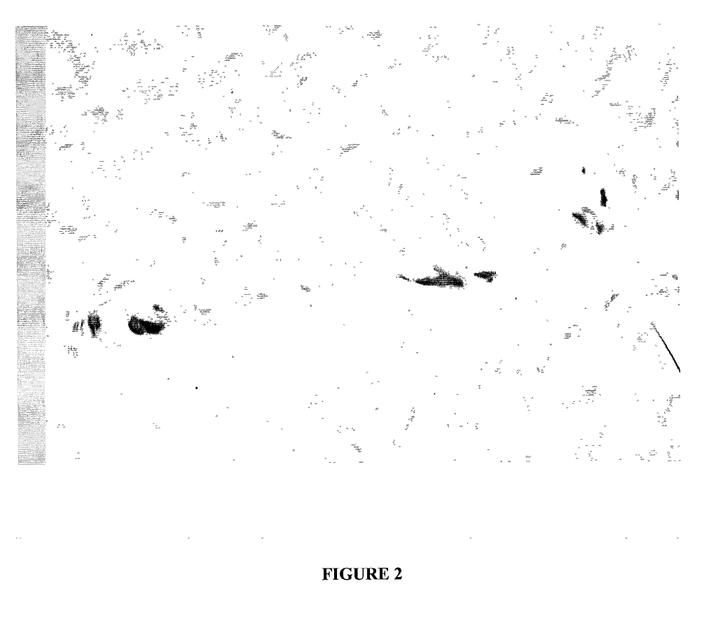

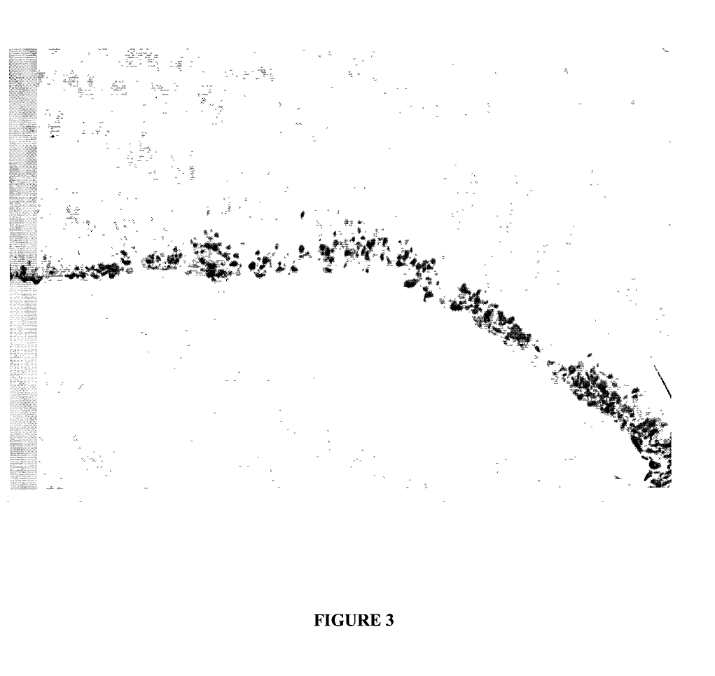

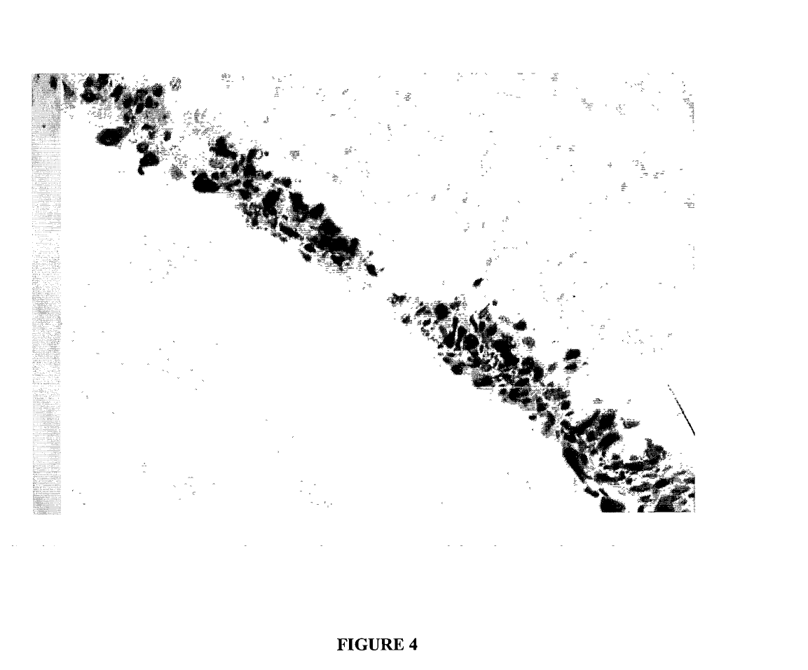

Analyses demonstrated that gene transfer was enhanced in atheromatous versus normal iliac artery. All cells of the arterial wall, i.e., endothelial, neointimal, and smooth muscle, were transduced by the adenovirus. There was no cellular inflammatory response due to the catheter-mediated gene transfer. Representative pictures of arterial gene transfer with channeled balloon and e-Med catheters are shown in FIGS. 1-4.

Adenoviral gene delivery to the vessel wall was systematically superior, in terms of homogeneity and efficiency, with the use of percutaneous e-Med microporous catheters versus annular balloon or channeled balloon catheters (i.e., under clinically-relevant conditions such as the short delivery time of 2 minutes). This was observed in both normal and atheromatous rabbit iliac artery models of stenosis. Viral particles penetrated arterial tissue and transduced endothelial, neointimal, and smooth muscle cells with high efficiency. Therefore, we believe that the e-Med microporous balloon will be suitable for the delivery of therapeutic adenoviral solutions.

### EXAMPLE 2

### Adenoviral-Mediated β-Gal Gene Transfer in a Porcine Model of Coronary Artery Restenosis

1. Summary

The ability of four local delivery devices to deliver a marker gene, β-gal, encoded by an adenoviral vector (AV_{1.0}CMVβ-gal/10¹² virus particles (vp)/artery), immediately before stent placement in the atherosclerotic left anterior descending (LAD) and left circumflex (LCX) coronary arteries of hypercholesterolemic pigs was evaluated. The possible influence of arterial branches on the transfer of the marker gene into the arterial wall also was evaluated. The devices tested were: (1) a Nycomed annular balloon catheter (Nycomed Medical Systems, Paris, France); (2) an e-Med Microfuse® catheter (e-Med, St. Paul, Minn.); (3) an IVT Infiltrator® catheter (Interventional Technologies, San Diego, Calif.); and (4) a Boston Scientific channeled balloon catheter (Boston Scientific, Natick, Mass.). The relative efficacy of gene transfer was initially determined by semi-quantitative analysis of the intensity of apparent staining of arteries with β-gal following incubation in X-gal solution for 18 hr. Quantitative measurement of the area of staining was also performed.

Microscopic quantification of cells staining for β-gal was used to define the absolute efficacy of gene transfer.

In general, while a relatively high degree of apparent staining was observed in both the LAD and LCX arteries across all treatment groups, quantification of cells staining for β-gal suggested that, overall, only low transfer of the marker gene was achieved, and that the e-Med Microfuse® catheter transferred the gene more effectively than the other devices. Statistical analysis of the number and distribution of arterial branches along the LAD and LCX arterial segments exposed to the marker gene detected significantly more branches in the LAD arterial segments than in the LCX arterial segments. ANOVA, however, found no difference in the number of branches per arterial segment when they were grouped by local delivery device.

2. Materials and Methods

2.1 Animals

A porcine model of restenosis, which incorporates plaque creation and stent deployment in the LAD and LCX coronary arteries of hypercholesterolemic swine, was used. Plaque creation was caused by a combination of atherogenic diet, acute balloon over-stretch, and denudation of the artery. This process achieved about 50% arterial stenosis within 4-5 weeks. Four to nine weeks post plaque creation (after acute artery over-stretch), a vascular stent, sized to approximate the artery area, was expanded at the site of pre-established lesion. This procedure has been shown to result in neointima formation and restenosis of the artery.

Thirty adult male, Yucatan minipigs (31.8±1.5, range: 21.1-57.0 kg, Sinclair Research Center, Columbia, Mo.) were used for this study. Twenty-eight pigs were randomly assigned to one of four treatment groups, which differed only by the choice of local delivery device, and received the adenovirus encoded with the β-gal gene. Two pigs served as controls and received only buffer.

Three to five days before the initial procedure, and continuing throughout the study, pigs received an atherogenic diet (20% saturated fat, 2% cholesterol, Dyets, Bethlehem, Pa.) and aspirin (Ascriptin, 250 mg, bid).

At the time of balloon over-stretch, the animals received an initial 3,000 IU of heparin as an i.v. bolus followed by 1,000 IU i.v. every 20 minutes. A 0.95 mg/min lidocaine infusion was started and maintained for the duration of the procedure. Nitroglycerin was administered at 287 μg/min until a 5 mm decrease in blood pressure was observed; it was then decreased to 116 μg/min and maintained throughout the experiment. The arterial vasculature was accessed by a right lateral neck incision and exposure of the right carotid artery. A blood sample was obtained for determination of arterial blood gases, pH, ion concentration (I-stat, Sensor Devices, Inc., Waukesha, Wis.), and cholesterol level (Cholestrak, ChemTrak, Sunnyvale, Calif.).

With fluoroscopic guidance, an 8F Guide catheter (FCR3.5, SCIMED) was advanced to the coronary ostia and positioned so that the LAD artery was engaged. A 0.014″ percutaneous transluminal coronary angioplasty (PTCA) guide wire was then advanced into the artery and an intravascular ultrasound (IVUS) catheter (Ultracross, 30 MHz, SCIMED) was advanced to the end of the wire. IVUS studies were performed utilizing an automatic withdrawal rate of 0.5 mm/sec. Imaging runs were performed over a distance of 5 cm. The IVUS images were continuously recorded and stored on 0.5″ high-resolution VHS video tapes. After recording the imaging run, the IVUS transducer was again advanced to the start point and an angiogram of this position was recorded. Angiograms were subsequently recorded every 1 cm over the 5 cm imaging distance for the purpose of accurately positioning the PTCA balloon. Arterial lumen measurements were made every 5 mm for 2 cm along a segment of artery which was relatively devoid of branches. The average lumen area along this segment was determined, and a balloon diameter was selected that could achieve a calculated 1.8×over-stretch of this area.

Using the angiograms as a guide, a non-compliant PTCA balloon (NC Express Plus, SCIMED) was advanced to the desired segment of artery and was expanded to the calculated diameter for 20 sec. The balloon was then deflated and the artery was allowed to rest for 1 min. This cycle was repeated twice and was followed by three gentle rubs of the endothelium with the balloon inflated to a diameter that produced slightly less than a 1.8× over-stretch of the artery. The balloon and guide wire were withdrawn and the guide catheter was repositioned so that the LCX artery could be engaged, and the entire procedure was repeated. The carotid artery was ligated and the neck incision was closed in two layers, using 3-0 vicryl for each layer.

Animals were placed in an intensive care unit until they recovered from anesthesia, and were then returned to their home cages. Three days prior to the gene delivery procedure, pigs received ticlopidine (250 mg, bid), which was continued for a total of 6 days.

At 39±1 (range: 29-62) days after the initial arterial injury, pigs were brought back into the catheterization lab and under the conditions described above, the created stenosis in the LAD and LCX coronary arteries was located. An IVUS run was performed as described above. The artery and lumen cross-sectional areas (CSA) along a 2 cm length of stenotic artery were measured, from which the artery and lumen diameters were calculated. This allowed selection of the delivery device size and a balloon/stent catheter for stent deployment. The lumen area measurements determined the size of the delivery device, and the artery area measurements determined the size of the balloon/stent.

Under sterile conditions, the local delivery catheter was filled with the marker gene and contrast solution as described below, and the device was used in accordance with each manufacturer's instructions. A new delivery catheter was used for each artery. After delivery of the marker gene, a balloon expandable NIR Stent (NIR PRIMO, SCIMED) pre-mounted on a non-compliant balloon was advanced to the site of gene delivery and expanded to approximate the artery area. An angiogram was obtained to confirm successful stent deployment.

The animals were again placed in an intensive care unit until they recovered from anesthesia, and were then returned to their home cages. Three days following gene delivery and stent deployment, animals were euthanized with an overdose of pentobarbital and the heart was removed. The LAD, LCX, and right coronary arteries were dissected from the underlying myocardium, and prepared for histological analysis as described below.

2.2 Preparation of the Local Delivery Catheters for Marker Gene Delivery

A working solution of adenovirus (10¹² vp) was prepared by diluting 200 μl of stock solution (AV_{1.0}CMVβGal/V143 in aliquots of 5×10¹² virus particles (vp)/ml) with 300 μl of PBS buffer solution (pH 7.4 containing 10% (v/v) glycerol and 0.1% (v/v) 0.5M MgCl₂). The adenovirus vector containing the β-gal marker gene was sandwiched between 2 columns of 50% contrast media solution. Under sterile conditions, a 30-inch extension set (Abbott Laboratories, North Chicago, Ill.), fitted at one end with a 3-way stopcock, was filled with 50% contrast media solution. A 6-inch extension set (Maxxim Medical, Athens, Tex.), fitted at each end with a 3-way stopcock, was filled with the adenoviral vector as follows. With the distal stopcock positioned so that the tubing was opened to air, 500 μl of the adenoviral solution were carefully loaded into the tubing until a small air bubble was observed in the hub of the proximal stopcock. The distal stopcock was then positioned so that the tubing was closed to air, and the long extension set containing the contrast media was attached to the proximal stopcock, ensuring that all the air was expelled from the stopcock. The dead-space volume of a local delivery catheter was then determined by slowly filling the catheter with 50% contrast media. The volume of catheter dead-space plus the 500 μl of adenovirus solution comprised the total volume of solution infused into the artery. The adenovirus end of the extension set combination was then secured to the local delivery catheter, and the unit was then taken to the catheterization suite for administration to the animal. Infusion times and/or balloon inflation pressures were set according to the manufacturer's instructions for each delivery device.

2.3 Preparation of Arteries for Histology

Three days following gene transfer, the animals were euthanized with an overdose of sodium pentobarbital, and the hearts were removed. The LAD, LCX, and right coronary arteries were carefully removed and placed in cold (4° C.) 3.7-4.0% formaldehyde in Dulbecco's PBS (DPBS) for 3 hr. The stents were carefully removed and the arteries were placed in a 20% sucrose solution in DPBS. The tissues were stored in this solution at 4° C. for up to 30 hr before being stained for β-gal.

In preparation for staining, the arteries were subjected to a series of washes in 2 mM MgCl₂/DPBS and were then placed in a 2 mM MgCl₂/DPBS permeabilization solution containing 0.01% (final conc.) deoxycholic acid and 0.02% (final conc.) Nonidet P-40 for 30 min at 4° C. The tissues were stained in X-gal solution for 18 hr at 37° C. with constant agitation. β-gal catalyzed the conversion of the X-gal to an insoluble blue precipitate, thus staining blue those tissues that expressed the enzyme.

Following incubation in the staining solution, the arteries were rinsed twice with DPBS, and photographed for semi-quantification of β-gal staining. All arteries were photographed after staining using EZ-Trac, Inc. software on a Dynex 386-25 PC with a Javelin MDS Solid State CCD camera. Images were captured each week from the tissues that were collected and stained that week. A metric ruler was placed in each image field for area calibration when the area of vascular staining was determined. After image capture, tissues were returned to 3.7-4.0% formaldehyde in DPBS and stored at 4° C. until microscopic determination of β-gal expression was performed in paraffin sections. Histological sections (5μ) were routinely counter stained with Nuclear Red. Eight sections were cut from each stented segment of the artery, and four views from each section were analyzed. Microscopic determination of β-gal expression was assessed in the arterial wall and in the perivascular tissues (small vessels, fibroblastic cells, macrophages, and myocardial muscle cells).

2.4 Analysis of Delivery Device Balloon Diameter to Lumen Diameter Ratios, the Number of Arterial Branches, and Relative Distribution of Branches in Treated Segments of LAD and LCX Coronary Arteries.

The delivery device balloon diameter to lumen diameter ratio for each treated artery was determined. Ratios were calculated by dividing the expanded balloon diameter by the lumen diameter. To determine if branches along the treated segment of the artery had a role in the uptake of the marker gene by the arterial wall, the number of branches in each treated segment of the LAD and LCX coronary arteries was counted. The branches were counted by viewing that segment of the IVUS tapes corresponding to the position of the delivery device balloon as determined by the length of the balloon. The relative position of the branches counted in the LAD arteries was subsequently determined. To perform this analysis, the treated segment of the artery was divided into two halves, a distal half and a proximal half. Then, using the length of the transfer region of the balloon as the length of the treated segment, the position of each branch was normalized relative to the total length of the treated area and expressed as a percentage of the treated length. Those branches positioned from 0-50% were placed in the distal half and those positioned from 51-100% were placed in the proximal half. The number of branches falling in each half was then expressed as a percentage of the total number of branches counted in that artery.

2.5 Statistics

Data are presented as the mean±sem. Student's t-test for unpaired data was used to test for differences between groups. ANOVA was used to test for differences within groups. Correlations were performed by linear regression using the least squares method. Results were considered statistically significant at p<0.05 (two-tailed).

3. Results

3.1 Summary of Delivery Device Characteristics.

A summary of some of the more pertinent delivery device characteristics supplied by the manufacturers is presented in Table 1. Table 2 presents data comparing parameters, such as balloon inflation pressure, volume of infusate, and the time of infusion, generated during use of the different delivery devices in this study. Table 2 also provides a summary of the microscopic quantification of β-gal staining in the LAD and LCX coronary arteries.

3.2 Intensity of Apparent Staining.

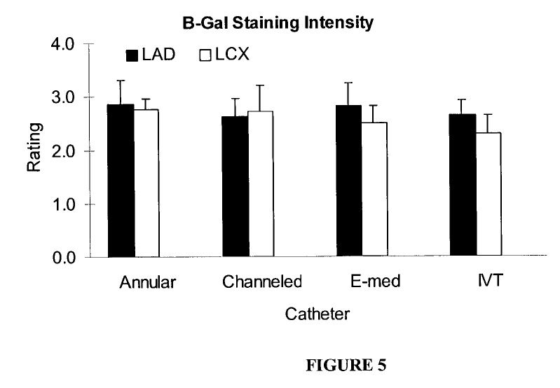

A semi-quantitative rating scale of 0-4 was devised to rate the intensity of apparent β-gal staining of the arteries. This is the staining observed in arteries before they were embedded in paraffin. Photographs of arteries from a given staining run were analyzed together. Arteries demonstrating no apparent staining were rated 0, while those arteries demonstrating the greatest amount of apparent staining were rated 4. The photographs were rated by three independent observers who were unaware of the treatment group to which the arteries belonged. The scores were averaged and are presented in FIG. 5. In general, a relatively high degree of apparent staining was observed in both the LAD and LCX arteries across all treatment groups, and no significant difference (p=0.96) was seen among the different groups. No staining was evident in the right coronary artery from any animal.

3.3 Quantification of the Area of Apparent β-Gal Staining.

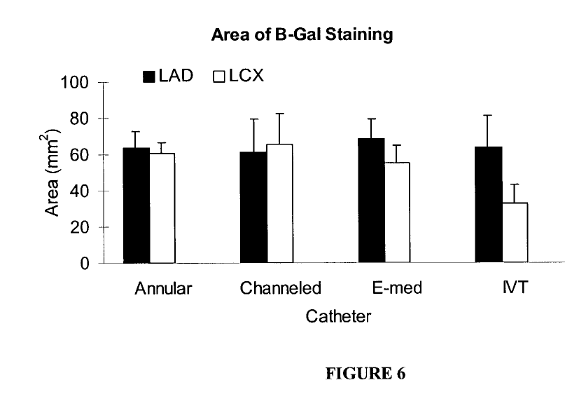

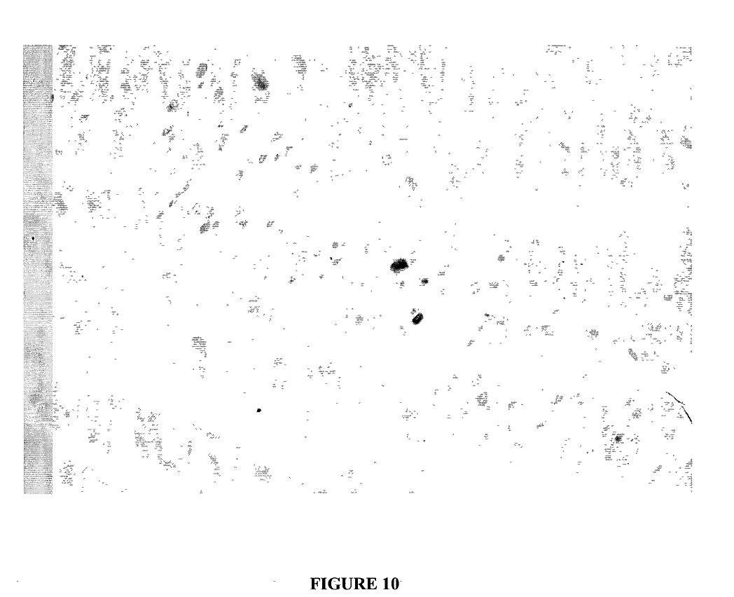

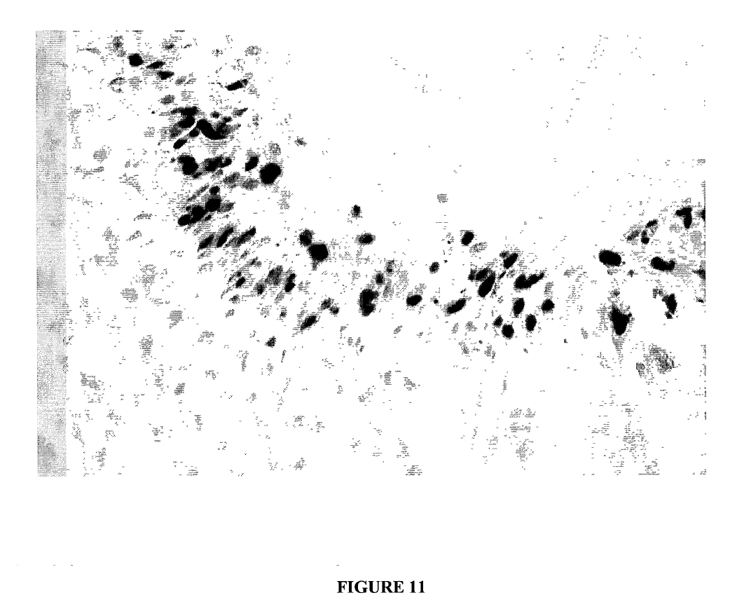

Photographs of the samples were used to measure the area of staining. Using the metric ruler recorded in each image, the system was calibrated to measure in millimeters, and the area of visible blue staining was traced. The area measurements were averaged by artery for each treatment group, and are presented in FIG. 6. For the LAD artery, the area of staining ranged from 61.4±18.0 to 68.5±10.7 mm², while for the LCX artery, the area of staining ranged from 32.8±10.4 to 65.7±16.9 mm². There was no statistically significant difference (p=0.67) in the area of artery stained between the LAD and LCX arteries, among any of the devices tested. Representative pictures of arterial gene transfer with channeled balloon and e-Med catheters are shown in FIGS. 10 and 11.

3.4 Microscopic Quantification of β-Gal Expression.

Recombinant adenovirus, containing a nucleus-localized variant of β-gal (LacZ β-gal) was used to quantitate gene transfer. In the arterial wall, LacZ β-gal expression was detected in endothelial cells, neointimal cells, and necrotic cells of undetermined character. LacZ β-gal expression also was observed in the perivascular tissues where it was localized to macrophages, fibroblastic cells, and myocardial cells.

The e-Med catheter transfected statistically significant more arteries than the other catheters tested (Table 5). More than 70% of the coronary arteries were transduced using the e-Med catheter, whereas 38%, 17% and 20% of arteries were transduced with the annular balloon, channeled balloon, and the Infiltrators® catheters, respectively. In addition, the average number of cells staining positive for β-gal in the transfected arterial segments was significantly greater for the e-Med catheter than for the other catheters. The data indicate superior gene transfer in the arterial wall using the e-Med Microfuse® catheter.

3.5 Cholesterol Levels.

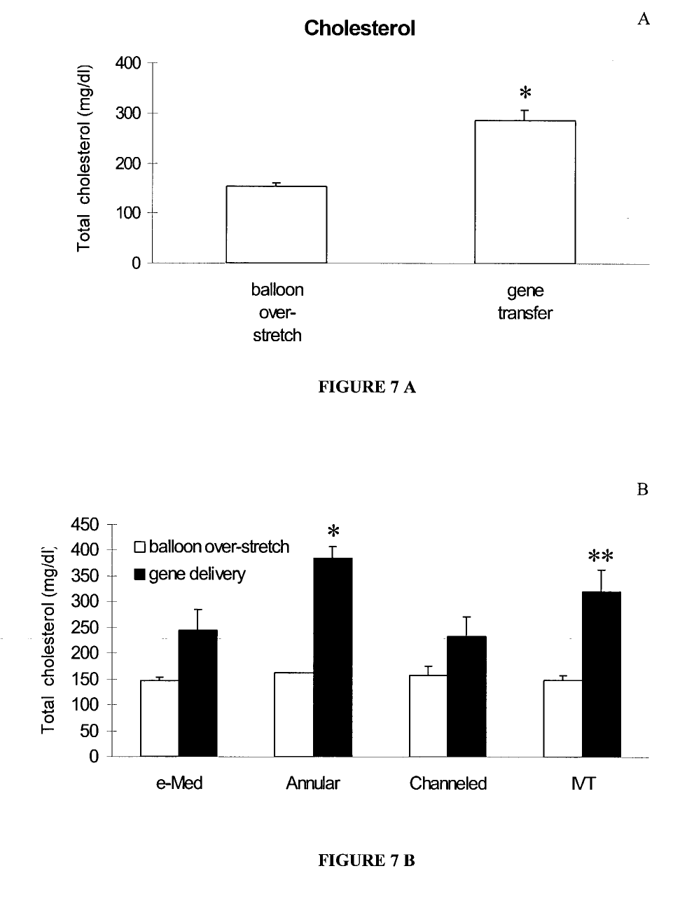

The mean total cholesterol level of the pigs at the time of balloon over-stretch was 153.8±5.2 mg/dl. At the time of gene delivery and stent deployment, the mean total cholesterol had increased significantly (p<0.001) to 284.8±22.1 mg/dl (FIG. 7A). ANOVA showed that the total cholesterol levels at the time of plaque creation did not differ significantly among the treatment groups. However, at the time of gene transfer and stent expansion, significant differences were observed. In the pigs treated with the annular balloon, cholesterol was significantly higher (p=0.03) than in the pigs treated with the Microfuse® or channeled balloons. Likewise, the average cholesterol level in the pigs treated with Infiltrator® balloon was significantly greater (p=0.03) than that in the pigs treated with the channeled balloon (FIG. 7B). When the relative changes in cholesterol, from the time of plaque creation to gene transfer, were analyzed, a significant difference (p=0.03) was observed between only the annular group (245.5 point increase) and the channeled balloon group (47.7 point increase). Since the arteries from either of these groups demonstrated very little staining, it is unlikely that differences in cholesterol accounted for the differences in arterial staining observed between the e-Med group and the other groups.

3.6 Summary of Balloon Diameter to Lumen Diameter Ratios.

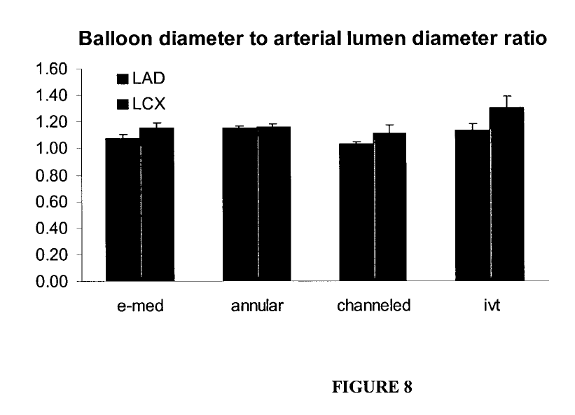

An analysis of the balloon diameter to lumen diameter was performed in order to eliminate the possibility that a difference in the balloon diameter to lumen diameter ratios or, alternatively, the amount of dilation experienced by the artery during gene delivery, might have affected the uptake of the marker gene by the arterial wall. The arteries were grouped according to the local delivery device with which they were treated. No significant difference (p=0.09) in the balloon diameter to lumen diameter ratio among the four groups was found (FIG. 8). The balloon to lumen diameter ratios were then subjected to linear regression analysis relative to arteries staining positive for β-gal. No correlation was found between arteries staining positive for β-gal and the balloon to lumen diameter ratios. The data are presented in Table 3.

3.7 Summary of Arterial Branch Analysis.

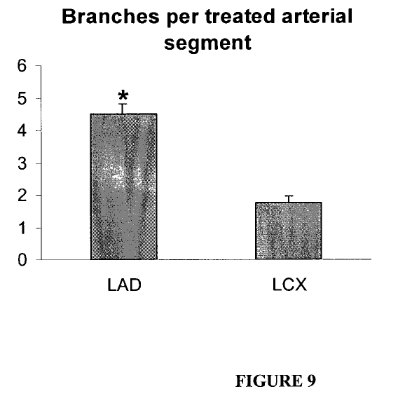

To ensure that the number of branches, or their distribution, was not different for any of the arteries subjected to gene transfer, an analysis of branch number and their relative distribution in each treated arterial segment was undertaken. The branches were counted by viewing that segment of the IVUS tapes corresponding to the position of the delivery device balloon as determined by the transfer region of the balloon. A significant difference (p<0.001) was found between the number of branches found in the LAD artery and those counted in the LCX artery (FIG. 9). Table 4 shows the data from an analysis of the relative position of the branches counted in the LAD arteries. No significant difference was observed in either the number of branches, normalized for the length of transfer region (p=0.1, data not shown), or the relative position of branches (p=1.0) among any of the treatment groups.

These results show that the e-Med Microfuse® catheter more effectively transferred the gene than did the other three devices. In addition, the e-Med catheters allowed the most complete cellular transduction along the arterial segment corresponding to the length of the balloon.

Although the total cholesterol level can influence the composition of an atherosclerotic lesion in pigs (Recchia et al., Arterioscler. Thromb. Vasc. Biol. 15:924-929, 1995), it seems unlikely that lesion composition, as projected from cholesterol levels, could explain the observed differences in artery staining. At the time of plaque creation (acute balloon over-stretch), no significant difference in the cholesterol level among the four groups was evident. At the time of gene transfer, the mean total cholesterol of the pigs in the annular group was 407 mg/dl, which was significantly higher than that of the pigs in either the e-Med group or the channeled group (FIG. 7B). However, the total number of arteries staining positive for β-gal was markedly higher in the e-Med group compared with both the annular and channeled groups. While the cholesterol level was significantly higher in the annular group than in the channeled group, the number of arteries staining positive for β-gal did not differ between these two groups (Table 2).

The data also indicate that differences in balloon to lumen ratios did not affect transfer of the gene. The degree of arterial injury is directly linked to the relative amount of acute balloon over-stretch imposed upon a naive artery (Bonan et al., Am. Heart J. 126:1330-1340, 1993). Although the extent to which this is true in a stenosed artery is debated (Carter et al., J. Am. Coll. Cardiol. 27:1270-1277, 1996), re-injury of the artery during gene transfer could alter arterial uptake of the gene. Therefore, during gene transfer, it is important to select a delivery device balloon size that approximates the lumen diameter to minimize arterial wall injury. An analysis of the delivery device balloon diameter to arterial lumen diameter ratios showed no significant differences among any of the groups.

In summary, the utilities of four local delivery devices (Nycomed “annular balloon” catheter, e-Med “Microfuse®” catheter, IVT “Infiltrator®” catheter, and Boston Scientific “channeled balloon” catheter) were compared for gene transfer into the cells of the arterial wall of injured, atherosclerotic LAD and LCX coronary arteries of hypercholesterolemic swine. Of the four devices, the e-Med Microfuse® catheter resulted in the greatest number of arteries staining positive for β-gal. These results clearly show that the combination of e-Med Microfuse® catheter and adenovirus comprising a gene provides an advantageous system for the efficient transfection of arterial cells. Naked DNA would not achieve a comparable level of transduction.

TABLE 1
Delivery Device Characteristics

| | | | Working | Transfer | | |
|---|---|---|---|---|---|---|
| Delivery | | Flow | length | region | | # of |
| Device | Type | thru | (cm) | (cm) | Pore size | pores |
| e-Med | intra- | no | 2.0 | 1.7 | <1 μ | >10⁶ |
| Microfuse ® | luminal | | | | | |
| Boston | intra- | no | 2.0 | <2.0 | 100 μ | 9 |
| Scientific | luminal | | | | | |
| Channeled | | | | | | |
| Nycomed | intra- | yes | 2.0 | 1.0 | annular | 0 |
| Annular | luminal | | | | chamber | |
| IVT | intra- | no | 1.5 | >1.5 | 100 μ | 21 |
| Infiltrator ® | mural | | | | | |

TABLE 2
Artery staining and device characteristics for gene delivery.

| | + staining | | Inflation | | Volume | | Infusion | | Length of | |
|---|---|---|---|---|---|---|---|---|---|---|
| | for β-gal | | pressure (atm) | | infused (ml) | | rate (ml/hr) | | infusion (sec) | |
| Delivery device | LAD | LCX | LAD | LCX | LAD | LCX | LAD | LCX | LAD | LCX |
| e-Med Microfuse ® | 7 | 5 | 3.5 ± 0.5 | 3.5 ± 0.5 | 1.3 ± 0 | 1.8 ± 0.4 | 108 ± 13 | 152 ± 22 | 45 ± 5 | 44 ± 6 |
| Boston Scientific Channeled | 3 | 0 | 4.5 ± 0.7 | 4.5 ± 0.7 | 0.8 ± 0 | 0.7 ± 0 | 27 ± 3 | 30 ± 2 | 90 ± 0 | 90 ± 0 |
| Nycomed Annular | 2+ | 0 | 10.0 ± 0 | 10.0 ± 0 | 0.8 ± 0 | 0.8 ± 0 | 12 ± 0 | 12 ± 0 | 250 ± 3 | 248 ± 2 |
| IVT Infiltrator ® | 1 | 2 | 2.0 ± 0 | 2.0 ± 0 | 1.0 ± 0 | 1.0 ± 0 | 122 ± 1 | 118 ± 2 | 30 ± 0 | 30 ± 0 |

TABLE 3
Artery staining and balloon diameter to lumen diameter ratios.

| | Number of arteries | | Balloon diameter to lumen | |
|---|---|---|---|---|
| Delivery | staining + for β-gal | | diameter ratio | |
| Device | LAD | LCX | LAD | LCX |
| e-Med Microfuse ® | 7 | 5 | 1.08 ± 0.03 | 1.15 ± 0.04 |
| Boston Scientific | 3 | 0 | 1.04 ± 0.02 | 1.11 ± 0.07 |
| Channeled | | | | |
| Nycomed Annular | 2+ | 0 | 1.15 ± 0.02 | 1.16 ± 0.03 |
| IVT Infiltrator ® | 1 | 2 | 1.14 ± 0.05 | 1.31 ± 0.09 |

TABLE 4
Summary of the number of branches and their relative distribution in
LAD arteries. Analysis by local delivery device.

| | Total | | | | |
|---|---|---|---|---|---|
| Delivery | number of | Distal | Percentage | Proximal | Percentage |
| device | branches | branches | of total | branches | of total |
| e-Med | 3.2 ± 0.5 | 1.9 ± 0.2 | 60 ± 7 | 1.4 ± 0.4 | 40 ± 7 |
| Microfuse ® | | | | | |
| Boston | 4.4 ± 0.5 | 1.9 ± 0.3 | 43 ± 5 | 2.6 ± 0.4 | 57 ± 5 |
| Scientific | | | | | |
| Channeled | | | | | |
| Nycomed | 2.2 ± 0.5 | 1.0 ± 0.3 | 56 ± 16 | 1.2 ± 0.4 | 44 ± 16 |
| Annular | | | | | |
| IVT | 4/7 ± 0.4 | 2.2 ± 0.7 | 43 ± 13 | 2.5 ± 0.5 | 57 ± 13 |
| Infiltrator ® | | | | | |

TABLE 5
Gene transfer efficiency. Number of detected Lac Z positive cells
identified in all artery segments after adenoviral delivery with each balloon

| | # arteries | Arterial gene transfer |
|---|---|---|
| | with Lac Z | efficiency (# LacZ positive |
| Device | expression | cells in arterial segments) |
| e-Med (n = 17) | 12 | 41 +/− 19* |
| Annular (n = 13) | 3 | 3 +/− 1 |
| Channeled (n = 17) | 5 | 2 +/− 1 |
| Infiltrator ® (n = 13) | 3 | 1 +/− 1 |

*Signifies p < 0.05 compared with other balloons; values in parenthesis indicate number of arteries exposed to gene transfer catheter.

We claim:

1. A method for delivering a gene of interest to a vascular tissue, comprising:
(a) inserting into said vascular tissue a catheter in fluid communication with an inflatable balloon, wherein said inflatable balloon is formed from a microporous membrane; and
(b) delivering to said vascular tissue through said catheter a solution comprising a vector comprising the gene of interest.

2. The method according to claim 1, wherein said gene is expressed in said vascular tissue.

3. The method according to claim 1, wherein said vector is a viral vector.

4. The method according to claim 3, wherein said vector is an adenoviral vector.

5. The method according to claim 1, wherein said microporous membrane has pores sized from about 10 Å to about 1μ and a pore density from about 10⁴ pores/cm² to about 10¹¹ pores/cm².

6. The method according to claim 1, wherein said balloon is inflated.

7. The method according to claim 1, wherein said vascular tissue is an atherosclerotic artery.

8. The method according to claim 1, wherein said vascular tissue is proximal to and connected with an ischemic tissue or an ischemic organ.

9. The method according to claim 1, wherein said gene is a suicide gene.

10. The method according to claim 9, wherein said suicide gene is a thymidine kinase gene or a cytosine deaminase gene.

11. The method according to claim 1, wherein said gene encodes an angiogenic factor.

12. The method according to claim 11, wherein said angiogenic factor is fibroblast growth factor (FGF) or vascular endothelial growth factor (VEGF).

13. A method for treating a vascular disease, comprising:
(a) inserting into a vascular tissue a catheter in fluid communication with an inflatable balloon, wherein said inflatable balloon is formed from a microporous membrane; and
(b) delivering to said vascular tissue through said catheter a solution comprising a vector comprising a gene of interest, wherein said gene is expressed in said vascular tissue.

14. The method according to claim 13, wherein said vascular disease is restenosis.

15. The method according to claim 13, wherein said vascular disease is ischemic heart disease.

16. The method according to claim 13, wherein said vascular disease is peripheral artery disease.

17. The method according to claim 13, wherein said gene is a suicide gene.

18. The method according to claim 17, wherein said suicide gene is a thymidine kinase gene or a cytosine deaminase gene.

19. The method according to claim 13, wherein said gene encodes an angiogenic factor.

20. The method according to claim 19, wherein said angiogenic factor is fibroblast growth factor (FGF) or vascular endothelial growth factor (VEGF).

21. A method for treating an ischemic tissue, comprising:
(a) inserting into an artery connected with said tissue a catheter in fluid communication with an inflatable balloon, wherein said inflatable balloon is formed from a microporous membrane;
(b) positioning said catheter proximal to the ischemic tissue; and
(c) delivering to said artery through the catheter a solution comprising a vector comprising a gene encoding an angiogenic factor, wherein said gene is expressed in the cells of said artery.

22. The method according to claim 21, wherein said angiogenic factor is fibroblast growth factor (FGF) or vascular endothelial growth factor (VEGF).

23. A method for treating coronary artery disease, comprising:
(a) inserting into a coronary artery a catheter in fluid communication with an inflatable balloon, wherein said inflatable balloon is formed from a microporous membrane; and
(b) delivering to said coronary artery through the catheter a solution comprising a vector comprising a gene encoding an angiogenic factor, wherein said gene is expressed in the cells of said coronary artery.

24. The method according to claim 23, wherein said angiogenic factor is fibroblast growth factor (FGF) or vascular endothelial growth factor (VEGF).

25. A kit, comprising:
(a) a catheter in fluid communication with an inflatable balloon, wherein said inflatable balloon is formed from a microporous membrane; and
(b) a vector comprising a gene of interest.

26. The kit according to claim 25, wherein said gene of interest is a suicide gene.

27. The kit according to claim 26, wherein said suicide gene is a thymidine kinase gene or a cytosine deaminase gene.

28. The kit according to claim 25, wherein said gene of interest encodes an angiogenic factor.

29. The kit according to claim 28, wherein said angiogenic factor is fibroblast growth factor (FGF) or vascular endothelial growth factor (VEGF).
